(19) United States
(12) Patent Application Publication
(10) Pub. No.: US 2023/0321005 A1
(43) Pub. Date: Oct. 12, 2023

(54) PHYTONADIONE COMPOSITIONS

(71) Applicant: CIPLA LIMITED, Mumbai (IN)

(72) Inventors: Alagumurugan ALAGARSWAMY, Mumbai (IN); Tushar RAJYAGURU, Mumbai (IN); Sameer SONAWANE, Mumbai (IN); Rajdeep GHANWARY, Mumbai (IN)

(21) Appl. No.: 18/041,328

(22) PCT Filed: Aug. 11, 2021
(86) PCT No.: PCT/IN2021/050771

§ 371 Date: Feb. 10, 2023

(30) Foreign Application Priority Data
Aug. 11, 2020 (IN) 202021034467

(51) Int. Cl. A61K 47/18; A61K 47/12; A61K 31/122; A61K 9/00; A61K 47/26; A61K 9/107; A61K 47/10

(52) U.S. Cl. CPC A61K 31/122; A61K 9/0019; A61K 9/1075; A61K 47/10; A61K 47/12; A61K 47/183; A61K 47/26

(57) ABSTRACT

Long term storage stable injectable phytonadione containing liquid pharmaceutical formulations are disclosed. The compositions can include phytonadione or pharmaceutically acceptable salts thereof; an antioxidant; buffer, pH adjusting agent and a pharmaceutically acceptable fluid. The methods of preparing the formulation as well as methods of treatment of phytonadione deficiency diseases using the same are also disclosed.

### CROSS REFERENCE TO RELATED APPLICATIONS

This application claims priority to Indian Application No. 202021034467, filed 11 Aug. 2020 and entitled “PHYTONADIONE COMPOSITIONS” which is incorporated herein in its entirety.

### FIELD OF INVENTION

The present invention relates to stable injectable pharmaceutical formulation comprising Phytonadione. The invention relates to formulations comprising phytonadione and antioxidants, and specifically to the use of antioxidants for stabilizing the formulation of phytonadione. The invention further relates to the method for preparing the above formulation, and a method for treating coagulation disorders employing the above formulation.

### BACKGROUND OF THE INVENTION

Vitamin K includes a group of lipophilic, hydrophobic vitamins that are required for blood coagulation. Chemically, they are 2-methyl-1,4-naphthoquinone derivatives. Vitamin Kl, also known as phylloquinone, phytonadione or phytomenadione is known to be the active ingredient which exhibits significant anticoagulant activity.

Vitamin Kl, chemically identified as, 2-Methyl-3-[(2E,7R,1 1R)-3,7,1 1,15- tetramethyl-2-hexadecenyl]-1,4-naphthalenedione. In adults, gut bacteria normally produce significant amounts of usable vitamin K. However, patients undergoing treatment with large doses of antibiotics may develop vitamin K deficiency due to a reduced intestinal flora. In newborns, the gut has not yet been colonized with bacteria. Thus, phytonadione has been in use for several years in the United States and other countries. Phytonadione for parenteral administration has been known. Therefore, standard medical practice is to administer vitamin K injections prophylactically to prevent hemorrhagic disease of the newborn and to adults to prevent or treat vitamin K deficiency.

Phytonadione is 2 - methyl - 3 - phytyl - 1, 4 - naphthoquinone. Its empirical formula is C₃₁H₄₆O₂and its structural formula is

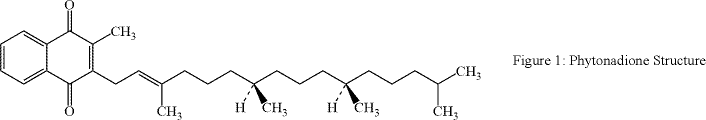 [SMILES: CC1=C(C/C=C(\C)CCC[C@H](C)CCC[C@H](C)CCCC(C)C)C(=O)c2ccccc2C1=O]

Phytonadione is known to be highly susceptible to oxidative and photolytic degradation to form multiple peroxide and epoxy impurities.

Some commercial products of phytonadione are available for the treatment of vitamin K deficiency. Vitamin K1 Injection (Phytonadione Injectable Emulsion, USP) of Hospira is a sterile, nonpyrogenic aqueous dispersion available for injection by the intravenous, intramuscular, and subcutaneous routes. Each millilitre contains phytonadione 2 or 10 mg, polyoxyl 35 castor oil 70 mg (also known as Koilliphor EL/ Etocas 35), dextrose monohydrate 37.5 mg in water for injection; benzyl alcohol 9 mg added as preservative; may contain hydrochloric acid for pH adjustment. pH is 6.3 (5.0 to 7.0).

Another commercially available formulation marketed by International Medicines company is Phytonadione Injectable Emulsion, USP, which is a yellow, sterile, aqueous colloidal solution of vitamin K1, with a pH of 3.5 to 7.0. It is available for injection by the intravenous, intramuscular, and subcutaneous route. Each 0.5 mL contains 1 mg phytonadione (Vitamin K1), 10 mg polysorbate 80, 10.4 mg propylene glycol, 0.17 mg sodium acetate anhydrous, and 0.00002 mL glacial acetic acid. Additional glacial acetic acid or sodium acetate anhydrous may have been added to adjust pH to meet USP limits of 3.5 to 7.0. The air above the liquid in the individual containers has been displaced by flushing with nitrogen during the filling operation.

Another commercial formulation, Konakion® MM 2 mg is available in ampoules as a Solution for injection or oral liquid. The ampoule contains the active ingredient phytomenadione 2 mg/ 200 µL in a mixed micelles (MM) solution (the micelles are composed of glycocholic acid and lecithin in an aqueous solution). The MM ampoule also contains sodium hydroxide, hydrochloric acid and water for injection. Konakion® MM 2 mg is approved for prophylaxis and therapy of vitamin K deficiency bleeding in newborns.

These marketed products of phytonadione need to be stored as per the special instructions such that the container containing the product should be in original carton to be always protected from light since Vitamin K1 is rapidly degraded by light. The marketed product should be stored in original carton until all the contents have been used. It is also mentioned on the label of marketed products that that even during dosage and administration of the formulation, it should be always protected from light. Thus, there is always a risk of degradation of phytonadione in the formulations leading to formation of undesirable impurities. Hence, it is crucial to protect the product from degradation at all the stages such as manufacturing, storage, dilution, handling, and administration.

In addition, marketed formulations containing polyethoxy fatty acid derivatives (Also known as Polyoxyl 35 castor oil or Kolliphor EL or Etocas 35) which are known to cause adverse effects and anaphylactic reactions while also causing neurological and nephrological toxicities. Cremophor EL is also a known detergent causing changes in blood viscosity, and erythrocyte aggregation.

Some attempts have been made to reduce the adverse events of commercially available products. US20190015356 discloses a pharmaceutical composition comprising Phytonadione in its (E) isomer at a concentration of from about 0.1 mg / ml to about 20 mg / ml; optionally a pH adjuster, has a pH of from about 3.5 to about 8.0, optionally, comprises a polysorbate at a concentration of less than about 1: 10 ratios of (E) isomer of phytonadione: polysorbate. The composition is substantially free or free of preservatives, antioxidants and chelating agents and benzyl alcohol. However, one complication is acknowledged in the specification that the relative pharmacologic activity of the two isomers is not precisely known. Also it is difficult to manufacture the formulation as it needs to isolate E isomer and Z isomer and maintain it in the desired form.

WO2011153513A2 discloses a nano emulsion composition comprising vitamin K at about 0.1 to 2% by weight, a phospholipid, and a triglyceride oil, wherein the phospholipid-to-vitamin K weight ratio is between 13: 1 and 25: 1 and the oil concentration is no more than 1% by weight of the nano emulsion. The composition is stored at 25° C. for 3 months or undergoes a freeze-thaw treatment and the oil droplets of said composition have an average diameter of less than 200 nanometers and said composition exhibits a light transmittance value at 600 nm or 750 nm of no less than 20%. However, none of the prior attempts address the issue of degradation of phytonadione and prevention of formation of impurities.

Without being bound to any theory, the oxidative degradation of product necessitates inclusion of overage of active substance in the formulation to compensate the loss of potency through degradation over shelf life. It is observed that the currently marketed products contain about 5-8% of overage to compensate the potency loss due to degradation through the shelf life.

Therefore, there is a need to provide stable formulations comprising phytonadione wherein the level of oxidative degradation products is controlled within the levels recommended by ICH guideline ICH Q3B (R2) Impurities in New Drug Products consistently during manufacture, dilution, handling, administration, and storage for longer time, avoid the necessity to add overages of active in the formulation, devoid of adverse effects and is easy to manufacture.

The inventors of the present invention evaluated the properties of phytonadione, its impurity profile, the excipients, and the interaction of excipients with phytonadione, formation of impurities at different conditions during and after the preparation of formulation, composition of parenteral phytonadione formulations, and the manufacturing process of phytonadione compositions. After rigorous experimentation, the inventors have provided such pharmaceutical formulation which fulfil the said requirements and overcome the disadvantages of prior attempts. The sterile, stable pharmaceutical formulation of present invention comprises of phytonadione, antioxidants and pharmaceutically acceptable excipients which can be stored for longer time without formation of undesirable impurities at an undesired level.

### OBJECT OF THE INVENTION

An object of the present invention is to provide an injectable pharmaceutical composition comprising phytonadione and pharmaceutically acceptable excipients.

Another object of the present invention is to provide a process of preparing an injectable pharmaceutical composition comprising phytonadione and pharmaceutically acceptable excipients.

Yet another object of the present invention is to provide an injectable pharmaceutical composition comprising phytonadione and pharmaceutically acceptable excipients which is stable when stored for long term without necessitating addition of overages in the formulation.

Another object of present invention is to provide an injectable pharmaceutical composition comprising phytonadione and pharmaceutically acceptable excipients which when stored for longer duration of time, will undergo less oxidative degradation and thus forming less oxidative degradation products.

Still one of the objectives of present invention is to provide an injectable pharmaceutical composition comprising phytonadione and pharmaceutically acceptable excipients for the treatment of hemorrhagic disease in newborn and adults caused by deficiency of vitamin K.

### SUMMARY OF THE INVENTION

According to one aspect of the present invention there is provided an injectable pharmaceutical composition comprising phytonadione and pharmaceutically acceptable excipients.

According to another aspect of the present invention there is provided a process of preparing an injectable pharmaceutical composition comprising phytonadione and pharmaceutically acceptable excipients.

According to yet another aspect of the present invention, there is provided an injectable pharmaceutical composition comprising phytonadione and pharmaceutically acceptable excipients which is stable when stored for long term .

According to still another aspect of the present invention, there is provided an injectable pharmaceutical composition comprising phytonadione and pharmaceutically acceptable excipients which when stored for longer duration of time, will undergo less oxidative degradation and thus forming less oxidative degradation products.

According to still another aspect of the present invention, there is provided a method of alleviating or treating hemorrhagic disease in newborn and adults caused by deficiency of vitamin K by administering an injectable pharmaceutical composition comprising phytonadione and pharmaceutically acceptable excipients.

### DESCRIPTION OF THE INVENTION

Unless defined otherwise, all technical and scientific terms used herein have the same meaning as is commonly understood by one of ordinary skill in the art to which this invention belongs. In the event that there is a plurality of definitions for a term herein, those in this section prevail unless stated otherwise.

It must be noted that, as used in this specification and the appended claims, the singular forms “a” “an,” and “the” include plural referents unless the context clearly dictates otherwise. Thus, for example, reference to ″a container″ includes one or more of such containers and reference to the″agent″ includes reference to one or more of such agents. In describing and claiming the present invention, the following terminology will be used in accordance with the definitions set forth below.

As used herein, RRT is calculated by dividing the retention time of the peak of interest by the retention time of the main peak. Any peak with an RRT < 1 elutes before the main peak, and any peak with an RRT > 1 elutes after the main peak.

The term “injectable” as used herein includes the acceptance of an ingredient by a drug regulating authority (e.g., the US FDA) permitting its use in an injection drug.

In certain embodiments, the present compositions are both chemically and physically stable. A composition is “chemically stable” if the drug, e.g., vitamin K, in the composition is not substantially chemically degraded after storage under appropriate conditions for at least one month. In certain embodiments, the concentration of the intact vitamin K in the composition is reduced by less than about 5% under appropriate storage conditions (e.g., at -20° C., 2-8° C., or at room temperature) for at least 6 months.

As used herein, a formulation is “physically stable” if it can be stored under appropriate conditions for at least 6 months without an increase in its micelle size by more than 100%, or without micelle/ globule aggregation. In certain embodiments, the average size of particles of a composition of the present invention does not increase by more than about 10%, 20%>, 25%, 30%, 40%, 50%, 75%, or 100% under appropriate storage conditions (e.g., at -20° C., 2-8° C., or room temperature) for at least 6 months.

For purposes of the present invention, “substantially free of impurities” shall be understood to include phytonadione-containing compositions in which the amount of individual oxidative degradation impurities is not more than about 1.0% and total impurities is not more than 3.0% of the sum of peak areas of all degradants, as calculated on a normalized peak area response (“PAR”) basis as determined by high performance liquid chromatograph (“HPLC”) at a wavelength of 227 nm, after a period of about 18 months at a temperature of from about 5° C. to about 25° C. The amount of impurities is further calculated as being based upon the original amount of phytonadione (or salt thereof) being present in the composition or formulation.

As used herein, the term “particulate - matter - free” or its grammatical equivalents refer to the state in which the composition meets the USP requirements for particulate matter in parenteral solutions or emulsions.

TABLE 1

The oxidative degradation products of Phytonadione

| | | |
|---|---|---|
| Degradant name | Chemical Structure | Chemical name |
| Vitamin K1 Epoxide (Transepoxy Impurity) | 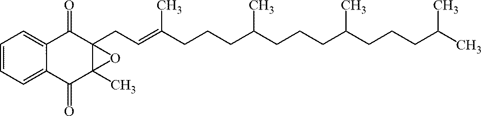 [SMILES: C/C(=C\CC12OC1(C)C(=O)c1ccccc1C2=O)CCCC(C)CCCC(C)CCCC(C)C] | 1a,7a-Dihydro-1a-methyl-7a-(3,7,11,15-tetramethyl-2-hexadecen-1-yl)-naphth[2,3-b] oxirene- |
| | | 2,7-dione; |
| Vitamin K1 Hydroperoxide | 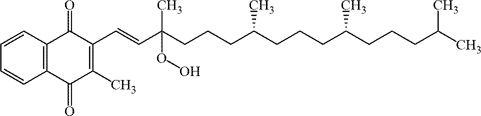 [SMILES: CC1=C(/C=C/C(C)(CCC[C@H](C)CCC[C@H](C)CCCC(C)C)OO)C(=O)c2ccccc2C1=O] | 2-((7R,11R, E)-3-Hydroperoxy-3,7,11,15-tetramethylhexadec-1-en-1-yl)-3-methylnaphthalene-1,4-dione |
| Vitamin K1 Hydroxy Impurity | 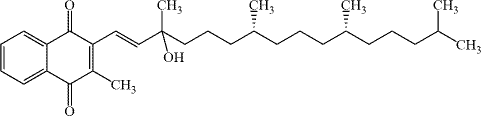 [SMILES: CC1=C(/C=C/C(C)(O)CCC[C@H](C)CCC[C@H](C)CCCC(C)C)C(=O)c2ccccc2C1=O] | 2-((7R,11R, E)-3-Hydroxy-3,7,11,15-tetramethyl-1-hexadecen-1-yl)-3-methyl-1,4-naphthalenedione; |
| Vitamin K1 Diol Impurity | 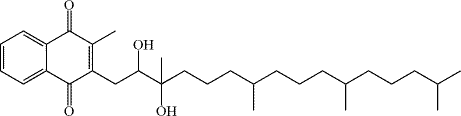 [SMILES: CC1=C(CC(O)C(C)(O)CCCC(C)CCCC(C)CCCC(C)C)C(=O)c2ccccc2C1=O] | 2-(2,3-dihydroxy-3,7,11,15-tetramethylhexadecyl)-3-methylnaphthalene-1,4-dione |

Preferably, the amount of any individual degradant in the inventive compositions does not exceed 1.0% PAR, more preferably does not exceed 0.5% PAR as determined by HPLC at a wavelength of 227 nm after storage periods of at least about 18 months at a temperature of from about 5° C. to about 25° C. In some aspects, the amount of time the inventive compositions demonstrate long term storage stability is at least about 18 months and preferably at least about 2 years when stored under the conditions described herein.

In some aspects of the present invention, the HPLC method includes the following: For purposes of the present invention, “long term storage” shall be understood to include at least time periods which are in excess of those observed when currently available phytonadione formulations are stored. In some preferred aspects of the invention, the time for which long term storage are contemplated include periods of at least about 18 months or longer.

The temperatures in which the liquid compositions are preferably kept are said to be either room temperature or less (i.e., about 25° C. or less). While not required, it is contemplated that storage can be further increased if carried out (optionally) under refrigerated conditions. For purposes of the present invention, “refrigerated conditions” shall be understood as being temperature below room temperature and preferably temperatures of less than about 10° C., preferably from about > 0° C. to about 10° C., more preferably from about 2 to about 10° C., yet more preferably about 3 to about 8° C., and still more preferably about 5° C. The term “refrigerated” conditions shall further be understood as including maintaining the composition at a substantially constant temperature and storage conditions within this range.

Phytonadione is insoluble in aqueous solvents. So, preparation of aqueous formulation of phytonadione involves suitable solubilization method which will keep the phytonadione solubilized in the formulation. Thus, formulation and process related issues become very significant. At the same time, considering the susceptibility of phytonadione to oxidative and photolytic degradation, controlling the impurity formation is important during and after the manufacturing of formulation.

The inventors of present invention have provided a stable pharmaceutical formulation comprising phytonadione. In the attempts to reduce the level of oxidative epoxy and peroxide impurities in the drug product, the inventors observed that some antioxidants were surprisingly helpful to reduce the impurity levels in the drug product. The experiments were carried out by formulating phytonadione formulations with incorporation of various antioxidants.

In accordance with an aspect of the invention, there are provided long term storage stable phytonadione containing liquid pharmaceutical compositions, comprising:
a) Phytonadione or a pharmaceutically acceptable salt thereof,
b) an antioxidant selected from the group consisting of L-Cysteine and Monothioglycerol and the salt forms thereof.
c) a surfactant to form micellar dispersion and
d) a pharmaceutically acceptable excipient.

The total impurities in the formulation of present invention resulting from the degradation of the phytonadione in the formulations is less than about 3.0% PAR, preferably less than 2.0% PAR as determined by HPLC at wavelength of 227 nm, after at least about 18 months of storage at a temperature of from about 5° C. to about 25° C., and thus have long term stability for at least the same period of time or longer.

Preferably, the phytonadione containing compositions demonstrate long term storage stability for at least about 2 years. In one embodiment, the amount of total impurities in the inventive compositions resulting from the degradation of the phytonadione is less than about 3.0% PAR, preferably less than 2.0% as determined by HPLC at a wavelength of 227 nm after at least about 2 years at a temperature of from about 5° C. to about 25° C. In another embodiment, the amount of total impurities is less than about 3.0% PAR, preferably less than 2.0% as determined by HPLC at a wavelength of 227 nm, after at least about 18 months of storage at a temperature of from about 5° C. to about 25° C.

As used herein the term “active ingredient” means all stereoisomers, including geometric isomers of vitamin K1 or phytonadione and their salts. It also refers to phytonadione obtained from both natural resources as well as obtained synthetically.

In some embodiment of the present invention, the phytonadione concentration is from about 0.5 mg/ml to about 20 mg/ml, preferably from about 1 mg/ml to about 15 mg/ml, and more preferably up to about 2 mg/ mL and 10 mg/ml. It will be understood that compositions containing any useful concentration within the ranges, i.e. 0.1, 1, 2, 3, 4, 5, 6, 7, 8, 9, 10 are contemplated. In alternative aspects, the amount of phytonadione is outside these ranges but the amounts will be sufficient for single or multiple administrations of dosages generally regarded as effective amounts.

For purposes of the present invention, a pharmaceutically acceptable fluid is a fluid which is suitable for pharmaceutical use such as aqueous solutions, water, saline, D5W, etc., optionally containing preservatives, tonicity, osmolality, buffers, etc., well known to those of ordinary skill in the art. Preferably, the pharmaceutically acceptable fluid includes water.

In several embodiments of the invention, the pharmaceutical compositions include water as a pharmaceutically acceptable fluid. In other embodiments of the invention, however, the pharmaceutical compositions include a mixture of propylene glycol (PG) and water. For example, in one embodiment, the pharmaceutical compositions include about 10% propylene glycol and about 90% water. Alternatively, the pharmaceutical compositions can include about 25% propylene glycol and about 75% water. In other embodiments, the pharmaceutical compositions can include up to about 75% propylene glycol. The amount of water and propylene glycol can be varied within the ranges, i.e. the ratio of water to propylene glycol in the pharmaceutical compositions can range from about 100% to 0% to about 25% to up to about 75%. Within this range, are phytonadione containing pharmaceutical compositions including up to about 75% propylene glycol and greater than about 25% water, and pharmaceutical compositions including about 50% water and 50% propylene glycol.

The term “antioxidants” as used herein preferably includes reducing agents or chelating agents that are safe to use in an injectable product. Reducing agents achieve their antioxidant effect by reacting with oxidizing agents in competition with test substance or by converting the oxidized test substance back to the original test substance in the reduced form. The reducing agents useful in certain embodiments of this invention include, but are not limited to, methionine, cysteine, thioglycerols, glutathione, ascorbic acid or salts thereof, ascorbyl palmitate, sodium metabisulfite, propyl gallate, butylated hydroxy anisole, butylated hydroxytoluene, tocopherol, histidine, amino acids or salts thereof, reducing sugars or any mixtures thereof. Chelating agents such as sodium citrate, tribasic (also referred to as trisodium citrate dihydrate), lactobionic acid, disodium ethylenediaminetetraacetic acid (EDTA) and tetrasodium EDTA can also use in formulation of present invention. Preferably, the antioxidants included in the formulation of present invention are those that contain thiol amino acids such as those selected from L- cysteine and thioglycerol. The amount of antioxidants present in the formulation of present invention is preferably 0.05 mg/mL to 10 mg/mL.

The phytonadione containing compositions according to several preferred aspects of the invention include a solubilized amount of antioxidant. For purposes of the present invention, “solubilized amount” shall be understood to include the saturation solubility concentration, where adding more antioxidants does not increase the concentration of the antioxidant in the final formulation of phytonadione containing compositions described herein. The amount of antioxidant included in the formulations described herein will vary somewhat, depending upon the antioxidant and phytonadione selected and other factors known to those of ordinary skill. Within this guideline, suitable antioxidant concentrations in the compositions will be from about 0.05 mg/ml to about 10 mg/ml, or the saturation solubility, whichever is higher in the final formulation. Concentrations of the antioxidant range from about 0.1 mg/ml to about 5 mg/ml are preferred, and concentrations of from about 0.2 mg/ml to about 1 mg/ml are more preferred.

Without meaning to be bound by any theory or hypothesis, metal ion induced oxidation of phytonadione is caused by metal ions leached from the surface of the metal or glass containers or from the elastomeric composition of the stopper in which phytonadione formulations were stored. The presence of reducing agents or chelating agents as antioxidants stabilizes phytonadione solution during long term storage.

In several embodiments of the invention the pH of the formulations is from about 3.5 to about 7, preferably from about 4.5 to about 6.5.

Some preferred long-term storage stable phytonadione containing compositions according to the present invention include:
a) Phytonadione.
b) Surfactant/solubilizing agent to form micellar dispersion
c) Antioxidant
d) Buffering agent/ pH adjusting agent.
e) A pharmaceutically acceptable fluid; wherein
f) The composition has a pH of pH 3.5-7.0

Preferably the formulation of present invention comprises the following:
a) Phytonadione.
b) Surfactant/solubilizing agent selected from polysorbate 80, polyoxyl 35 castor oil or mixed micelles comprising glycocholic acid and lecithin.
c) Antioxidant selected from L-cysteine hydrochloride, Monothioglycerol, L-Methionine, combination of BHT/BHA, Disodium Edetate or combinations thereof
e) Buffering agent and/or pH adjusting agent
g) A pharmaceutically acceptable fluid; wherein the composition has a pH of about 6.3

The formulation as described above have the preferred stability profiles described above, i.e. having less than about 1.0%, preferably less than about 0.5% of individual oxidative degradation products and less than 3% of total impurities, preferably less than 2.0% PAR, as determined by HPLC at a wavelength of 227 nm, after at least about 18 months of storage at a temperature of from about 5° C. to about 25° C.

A further aspect of the invention includes a kit and / or pharmaceutical container for holding the phytonadione-containing compositions described herein. As will be appreciated by those of ordinary skill, the kit will contain at least one pharmaceutically acceptable vial or container containing one or more doses of the phytonadione-containing formulations/compositions as well as other pharmaceutically necessary materials for storing and/or administering the drug, including instructions for storage and use, infusion bag or container with normal saline or D5W, additional diluents, if desired, etc. The diluent may also optionally include any known fluids capable of being included in sterile parenteral formulations. Such aqueous-based suitable fluids can include, for example, saline or dextrose if desired any as well of the known ancillary preservatives or excipients commonly found as part of parenteral formulations. In accordance with current FDA requirements, vials containing the inventive formulations contain well below the acceptable limits for particulate matter.

Thus, the vials contain:
Particles = ≥10 µm: Not more than 6000 per container (average)
Particles = ≥ 25 µm: Not more than 600 per container (average).

The compositions of the present invention can be packaged in any suitable sterile vial or container fit for the sterile storage for extended periods of time. Suitable containers can be glass vials, i.e. USP Type 1 vials, Sulphur treated vials, molded glass vials, and CZ resin vials, polypropylene or polyethylene vials, glass ampules, glass pre-filled syringes or other special purpose containers. Containers are of a size sufficient to hold one or more doses of phytonadione formulations.

A still further aspect of the invention includes methods of preparing the phytonadione compositions described herein. The methods include dissolving phytonadione or pharmaceutically acceptable salt thereof in a sufficient amount of a pharmaceutically acceptable fluid containing an antioxidant, a chelating agent, and optionally propylene glycol, and adjusting the pH to from about 6.3. in another aspect of the present invention, mmanufacturing process of formulation of present invention includes preparation of phytonadione concentrate phase and preparation of aqueous phase.

The phytonadione concentrate phase includes solubilizing/mixing active ingredient phytonadione in solubilizing agent/surfactant such as Polyoxyl 35 castor oil (Cremophor EL) or polysorbate 80. The aqueous phase is prepared by addition of excipient such as osmolality adjusting agent such as propylene glycol, dextrose, buffering agents such as sodium acetate & glacial acetic acid and antioxidant selected from Monothioglycerol, L-cysteine hydrochloride, L-methionine, BHA, BHT, Disodium Edetate or mixtures thereof. Final micellar dispersion is prepared by mixing phytonadione concentrate phase in aqueous phase, adjusting pH, and making up volume of this dispersion. Aqueous dispersions thus prepared had pH of about 3.5-7.0.

In a further aspect of the invention, there is provided a method of preventing the formation of phytonadione degradants in liquid phytonadione containing formulations during long term storage at room temperature.

Further optional steps in accordance therewith include aseptically transferring one or more pharmaceutically acceptable doses of the formulations into a suitable sealable container for storing the resultant solution in a sealed container at temperature of about 25° C. As a result of carrying out these steps, it is possible to control or substantially prevent the formation of impurities which otherwise occur with phytonadione containing formulations having individual oxidative degradation products less than about 1.0%, preferably less than 0.5% and total impurities less than about 3.0 %, preferably less than 2.0% PAR as determined by HPLC at a wavelength of 227 nm, after at least about 24 months of storage at a temperature of from about 5° C. to about 25° C.

In yet another aspect of the invention there are provided the compositions described herein are especially useful for treating blood coagulation disorders including (1) prophylaxis and therapy of haemorrhagic disease of the new born; (2) anticoagulant-induced prothrombin deficiency caused by coumarin or indanedione derivatives; (3) hypoprothrombinaemia due to antibacterial therapy; (4) hypoprothrombinaemia secondary to factors limiting absorption or synthesis of vitamin K; and (5) other drug-induced hypoprothrombinaemia. Acquired coagulation disorders are the result of conditions or diseases, such as vitamin K deficiency, liver disease, disseminated intravascular coagulation (DIC), or development of circulation anticoagulants. Patients undergoing anticoagulant therapies for the treatment of conditions, such as thromboembolism, can exhibit bleeding episodes upon acute administration of anticoagulants or develop haemorrhagic disorders as a result long term usage of such therapies. The compositions provided herein are useful for treatments for such bleeding episodes which typically include administration of procoagulants. The compositions provided herein can be used in treatments to control bleeding episodes in patients with acquired bleeding disorders due to anticoagulant treatments.

In an aspect of present invention, the formulation of present invention should be administered by the subcutaneous route preferably. In an embodiment, when intravenous administration is considered unavoidable for the administration of formulation of present invention, then the formulation should be injected very slowly, not exceeding 1 mg per minute.

### EXAMPLES

The following examples serve to provide further appreciation of the invention but are not meant in any way to restrict the effective scope of the invention.

### Example 1: Comparative Formulation Examples With Various Antioxidants

For the assessment of suitable antioxidant, formulations according to present invention were prepared comprising phytonadione, antioxidants, solubilizer and other excipients such as preservatives, tonicity, osmolality, buffers, and pharmaceutically acceptable fluid. The compositions are as follows.

TABLE 2

Comparative formulation examples with various antioxidants

| S.No. | Ingredient | Amount (mg/mL) | | | | | |
|---|---|---|---|---|---|---|---|
| | | Control formulation (without antioxidant) | Formula 1 (With monothiol-glycerol | Formula 2 (with L-Cysteine) | Formula 3 (with L-Methionine) | Formula 4 (with BHA/BHT) | Formula 5 (with EDTA) |
| 1. | Phytonadione | 2.0 | 2.0 | 2.0 | 2.0 | 2.0 | 2.0 |
| 2. | Monothioglycerol | - | 1.0 | - | - | - | - |
| 3. | L-Cysteine hydrochloride | - | - | 1.0 | - | - | - |
| 4. | L-Methionine | - | - | - | 1.0 | - | - |
| 5. | Combination of BHT/BHA | - | - | - | - | 0.15 BHT + 0.15 BHA | - |
| 6. | Disodium Edetate | - | - | - | - | - | 0.2 |
| 7. | Polysorbate 80 | 20.0 | 20.0 | 20.0 | 20.0 | 20.0 | 20.0 |
| 8. | Propylene glycol | 20.8 | 20.8 | 20.8 | 20.8 | 20.8 | 20.8 |
| 9. | Sodium acetate anhydrous | 0.34 | 0.34 | 0.34 | 0.34 | 0.34 | 0.34 |
| 10. | Glacial acetic acid | 0.00004 | 0.00004 | 0.00004 | 0.00004 | 0.00004 | 0.00004 |
| 11. | Glacial acetic acid and/or sodium acetate anhydrous | q.s. to pH 6.3 (3.5 to 7.0) | q.s. to pH 6.3 (3.5 to 7.0) | q.s. to pH 6.3 (3.5 to 7.0) | q.s. to pH 6.3 (3.5 to 7.0) | q.s. to pH 6.3 (3.5 to 7.0) | q.s. to pH 6.3 (3.5 to 7.0) |
| 12. | Water for Injection | qs to 1.0 mL | qs to 1.0 mL | qs to 1.0 mL | qs to 1.0 mL | qs to 1.0 mL | qs to 1.0 mL |

### Process of Preparation

1. Phytonadione concentrate phase was prepared by dissolving phytonadione in solubilizing agent/ surfactant, polysorbate 80.

2. Aqueous phase was prepared by addition of other water-soluble excipient such as propylene glycol, buffering agents such as sodium acetate & glacial acetic acid and antioxidant(s).

3. Micellar dispersion was prepared by mixing phytonadione concentrate phase in aqueous phase under continuous stirring, adjusting pH between 3.5 to 7.0 and finally the volume of this aqueous dispersion was made up to batch size.

4. Bulk aqueous dispersion was further aseptically filtered, filled, and sealed in suitable container system such as glass vial or prefilled syringe and stoppered by compatible rubber closure or sealed in Ampules.

5. The finished product as prepared in step 4 is to be stored/supplied in suitable secondary packaging material i.e., light protective tray/ mono carton.

### Stability Study

The stability of the product solutions filled in a prefilled glass syringe, stoppered with Flurotec laminated butyl rubber stopper was evaluated in accelerated stability conditions (40° C./75%RH). The critical quality attributes such pH, assay, level of oxidative degradation products of the compositions with the different anti-oxidants were assessed till a 6- month period.

TABLE 3

Stability study results of formulation with different antioxidant

| | Control Formulation | | Formula 1 | | Formula 2 (1 mg/mL | | Formula 3 (1 mg /mL | | Formula 4 (0.3 mg/mL | | Formula 5 (0.2 mg/mL | |
|---|---|---|---|---|---|---|---|---|---|---|---|---|
| | (Without antioxidant) | | (1 mg/mL Monothioglycer ol) | | L-Cysteine) | | L- Methionine) | | BHT-BHA) | | Disodium Edetate) | |
| Condition | Initial | 6 M 40° C./ 75%RH | Initial | 6 M 40° C./ 75%RH | Initial | 6 M 40° C./ 75%RH | Initial | 6 M 40° C./ 75%RH | Initial | 6 M 40° C./ 75%RH | Initial | 6 M 40° C./ 75%RH |
| Description | Clear, yellow solution | | Clear, yellow solution | | Clear, yellow solution | | Clear, yellow solution | | Clear, yellow solution | | Clear, yellow solution | |
| Assay (%) | 111.4 | 74.1 | 101.9 | 104.7 | 106.3 | 97.7 | 105.2 | 91.5 | 109.2 | 104.1 | 107.1 | 101.3 |
| Degradation products (%) | | | | | | | | | | | | |
| Transepoxy Impurity | <0.1 | <0.1 | <0.1 | <0.1 | <0.1 | <0.1 | <0.1 | 0.117 | <0.1 | 0.101 | <0.1 | 0.373 |
| Hydroperoxi de Impurity | <0.1 | 1.897 | <0.1 | 0.979 | ND | 0.544 | <0.1 | 0.354 | <0.1 | 0.585 | 0.37 | 3.892 |
| Hydroxy Impurity | ND | 3.648 | 0.18 | 0.112 | <0.1 | <0.1 | <0.1 | 5.487 | <0.1 | 0.641 | <0.1 | 1.525 |
| Diol Impurity | <0.1 | 0.283 | <0.1 | <0.1 | <0.1 | <0.1 | <0.1 | 0.1 | ND | <0.1 | <0.1 | 0.228 |
| Total Impurities | 0.619 | 9.95 | 0.377 | 1.79 | <0.1 | 1.45 | 0.176 | 6.64 | 0.62 | 1.56 | 1.07 | 6.83 |

Observation: It is observed that,
Antioxidants containing thiol functional group especially L-Cysteine and monothioglycerol were found significantly useful in minimizing the oxidative degradation products to below the desired NMT 1.0 % for the individual degradation products even in the accelerated stability conditions.
Additionally, combination of BHA & BHT system also found helpful for controlling the impurity profile.
In the assessed antioxidants, L-Cysteine and Monothioglycerol were preferred over the other antioxidants for controlling the oxidative degradation products.

### Example 2: Comparative Study of the Marketed Formulations

This example is to demonstrate the comparison of stability profile between formulations marketed formulation not comprising antioxidants and the formulation of present invention comprising antioxidants. The marketed formulations by Hospira Inc and International Medicines Systems Ltd. were assessed at accelerated stability condition (40° C./75%RH) for a period of 6 months. The data is compared with formulation containing 1-cysteine HCL as antioxidant.

TABLE 4

Comparative stability of marketed formulation and present invention with L-cysteine HCL monohydrate 1 mg/0.5 ml

| Formulation | IMS Product 1 mg/0.5ml | | Hospira Product 1 mg/0.5ml | | Hospira Product 10 mg/ml | | Formula 2 of present invention 1 mg/mL with L-Cysteine HCL) | |
|---|---|---|---|---|---|---|---|---|
| Condition | Initial | 6 Month 40° C./ 75%RH | Initial | 6 Month 40° C./ 75%RH | Initial | 6 Month 40° C./ 75%RH | Initial | 6 Month 40° C./ 75%RH |
| Description | Clear, yellow solution | | Clear, yellow solution | | Clear, yellow solution | | Clear, yellow solution | |
| Assay (%) | 106.7 | 97.1 | 105.6 | 99.7 | 107.9 | 104.0 | 106.3 | 97.7 |
| Degradation products (%) | | | | | | | | |
| Transepoxy Impurity | 0.441 | <0.1 | 3.526 | 3.340 | 2.956 | 9.935 | <0.1 | <0.1 |
| Hydroperoxide Impurity | 0.674 | 1.174 | 0.289 | 0.585 | Not Reporte d | 1.104 | ND | 0.544 |
| Hydroxy Impurity | <0.1 | 0.377 | 0.434 | | Not Reporte d | | <0.1 | <0.1 |
| Diol Impurity | <0.1 | <0.1 | ND | ND | ND | 1.77 | <0.1 | <0.1 |
| Total degradation products (%) | 1.33 | 2.08 | 4.628 | 4.434 | 4.492 | 11.446 | <0.1 | 1.45 |

Note: Total % impurities include total contributions from peaks at various RRTs.
ND: Not Detected

### Observation:

It was observed that concluded the marketed formulations that do not contain antioxidants showed a high level of oxidative degradation products compared to formulation of present invention containing preferred antioxidants.

### Example 3: Comparative Examples of Varying Concentration of L-cysteine HC1 Monohydrate

The following examples shows different concentration of L-cysteine HCL monohydrate that can be used for stabilization of Phytonadione drug product containing Polysorbate 80 as the surfactant.

TABLE 5

Comparative examples with varying antioxidant concentration

| Sr. No. | Ingredients | Amount mg/mL | |
|---|---|---|---|
| | | Formula 6 | Formula 7 |
| 1 | Phytonadione | 2.0 | 2.0 |
| 2 | L-cysteine HCL Monohydrate | 0.25 | 0.50 |
| 3 | Polysorbate 80 | 20.0 | 20.0 |
| 4 | Propylene glycol | 20.8 | 20.8 |
| 5 | Sodium acetate anhydrous | 0.34 | 0.34 |
| 6 | Glacial acetic acid | 0.00004 | 0.00004 |
| 7 | Glacial acetic acid or sodium acetate anhydrous | q.s. to pH 6.3 (3.5 to 7.0) | q.s. to pH 6.3 (3.5 to 7.0) |
| 8 | Water for Injection | up to 1.0 mL | up to 1.0 mL |

### Process of Preparation

1. Phytonadione concentrate phase is prepared by solubilizing/ mixing phytonadione in solubilizing agent/ surfactant such as polysorbate 80 to prepare micellar solution.

2. Aqueous phase is prepared by addition of excipient such as propylene glycol, buffering agent such as sodium acetate and glacial acetic acid and antioxidant, L-Cysteine hydrochloride.

3. The final dispersion was prepared by mixing phytonadione concentrate phase in aqueous phase under continuous stirring, adjusting pH between 3.5 to 7.0 and finally making up volume of this aqueous dispersion.

4. The dispersion prepared in step 3 was aseptically filtered and filled and sealed in suitable container system as described in invention i.e., Glass vial stoppered by compatible rubber closure or Ampule or prefilled syringe.

5. The finished product thus prepared in step 4 is to be stored/supplied in suitable secondary packaging material i.e., light protective tray/ monocarton.

### Stability Study

The stability of the product solutions filled in a prefilled glass syringe, stoppered with Flurotec laminated butyl rubber stopper was evaluated in accelerated stability conditions (40° C./75%RH). The critical quality attributes such pH, assay, level of oxidative degradation products of the compositions with the varying concentration of L-Cysteine were assessed till a 6- month period.

TABLE 6

Stability study results of formulation with different concentration of L-Cysteine HCl

| Formulation | Control Formulation (Without antioxidant) | | | Formula 6 (0.25 mg/mL L-Cysteine HCL) | | | Formula 7 (0.5 mg/mL L-Cysteine HCL) | | | Formula 2 (1 mg/mL L-Cysteine HCL) | | |
|---|---|---|---|---|---|---|---|---|---|---|---|---|
| Condition | Initial | 3 M 40° C./ 75% RH | 6 M 40° C./75 %R H | Initial | 3 M 40° C./ 75% RH | 6 M 40° C./ 75% RH | Initial | 3 M 40° C./ 75% RH | 6 M 40° C./ 75% RH | Initial | 3 M 40° C./ 75% RH | 6 M 40° C./ 75% RH |
| Diol Impurity (%) | <0.1 | 0.16 1 | 0.28 3 | <0.1 | <0.1 | 1.11 2 | 0.293 | 0.24 2 | 0.63 1 | <0.1 | <0.1 | <0.1 |
| Trans Epoxy impurity (%) | <0.1 | <0.1 | <0.1 | ND | <0.1 | 0.14 2 | 0.224 | 0.11 4 | 0.10 7 | <0.1 | <0.1 | <0.1 |
| Hydroperoxi de Impurity (%) | <0.1 | 5.53 1 | 1.89 7 | <0.1 | <0.1 | 0.17 4 | <0.1 | 0.17 6 | 0.25 9 | ND | <0.1 | 0.54 4 |
| Hydroxy Impurity (%) | ND | 1.37 | 3.64 8 | <0.1 | 0.63 2 | 1.13 8 | <0.1 | 0.43 8 | 1.31 5 | <0.1 | 0.18 4 | <0.1 |
| Total degradation products (%) | 0.619 | 8.04 4 | 9.95 | 0.24 | 2.08 6 | 6.44 | 0.78 | 1.92 5 | 4.76 | <0.1 | 0.45 3 | 1.45 |
| Assay (%) | 111.4 | 92.2 | 74.1 | 103.5 | 86 | 87.3 | 102.8 | 98.2 | 96.1 | 106.3 | 103. 8 | 97.7 |

Observation: It is observed that an increase in concentration of antioxidant provides a better protective action and the level of oxidative degradation products are minimized.

### Example 4: Formulation With L-Cysteine Hydrochloride as Antioxidant and Polyoxyl 35 Castor Oil as Solubilizing/Surfactant Agent

The following examples shows different composition containing Polyoxyl 35 castor oil as the surfactant.

TABLE 7

Examples of formulations containing Polyoxyl 35 castor oil

| Sr. No. | Ingredients | Function | Control formulation Formula 8 | Control formulation Formula 9 |
|---|---|---|---|---|
| 1 | Phytonadione | Active | 2 mg | 10 mg |
| 2 | L-cysteine HCl Monohydrate | Antioxidant | 1 mg | 1 mg |
| 3 | Polyoxyl 35 castor oil | Surfactant | 70 mg | 70 mg |
| 4 | Dextrose monohydrate | Tonicity adjuster | 37.5 mg | 37.5 mg |
| 5 | Benzyl alcohol | Preservative | 9 mg | 9 mg |
| 6 | Glacial acetic acid | pH adjusting agent | q.s. to pH 6.3 (3.5 to 7.0) | q.s. to pH 6.3 (3.5 to 7.0) |
| 7 | Water for Injection | Vehicle | up to 1.0 mL | up to 1.0 mL |
| 8 | Nitrogen | -- | -- | -- |

### Process of Preparation

1. Phytonadione concentrate phase was prepared by solubilizing/ mixing active ingredient phytonadione in solubilizing agent/surfactant such as Polyoxyl 35 castor oil (Cremophor EL) to prepare miscible solution.

2. Aqueous phase was prepared by addition of excipient such as tonicity agent Dextrose monohydrate, preservative Benzyl alcohol and antioxidant L-Cysteine hydrochloride monohydrate.

3. The final dispersion was prepared by mixing phytonadione concentrate phase in aqueous phase under continuous stirring, adjusting pH between 3.5 to 7.0 and finally making up volume of this aqueous dispersion.

4. Bulk aqueous dispersion prepared in step 4 was aseptically filtered and filled and sealed in suitable container system as described in invention i.e. Glass vial stoppered by compatible rubber closure or Ampule or prefilled syringe.

5. The finished product thus prepared is to be stored/supplied in suitable secondary packaging material i.e. light protective tray/ monocarton.

### Example 5: Composition of Phytonadione Injection of Present Invention at Large Scale

TABLE 8

The phytonadione injection of present invention can be reproduced at industrial level with below composition

| Sr. No | Name of Ingredients | Function | Formula 10 | |
|---|---|---|---|---|
| | | | Qty (mg/mL) | Qty (g/ 30L batch) |
| 1. | Phytonadione USP | Active ingredient | 2.0 | 60.00 g |
| 2. | Polysorbate 80 NF | Emulsifier | 20.0 | 0.60 kg |
| 3. | Propylene Glycol USP | Solvent | 20.8 | 0.624 kg |
| 4 | Sodium acetate anhydrous USP | Buffering agent | 0.34 | 10.2 g |
| 5 | Glacial acetic acid USP | pH adjusting agent | 0.00004 mL | 1.2 mL |
| 6 | L-Cysteine HCl monohydrate USP | Antioxidant | 0.25 | 7.5 g |
| 7 | Sodium acetate anhydrous USP | pH adjusting agent | q.s to adjust the pH 5.5 ± 0.2 | |
| 8 | Glacial acetic acid USP | pH adjusting agent | | |
| 9 | Water for Injection USP | Vehicle | q.s. to 1 mL | q.s. to 30 Litres |
| 10 | Nitrogen | Processing aid | q.s | q.s |

### Process of Preparation

1. Phytonadione concentrate phase was prepared by mixing phytonadione with surfactant, polysorbate 80.

2. Aqueous phase was prepared by addition of other water-soluble excipient such as propylene glycol, buffering agent such as sodium acetate, glacial acetic acid and L-Cysteine.

3. Micellar dispersion was prepared by mixing phytonadione concentrate phase in aqueous phase under continuous stirring, adjusting pH between 3.5 to 7.0 using diluted Sodium acetate and/or glacial acetic acid solution and finally the volume of this aqueous dispersion was made up to batch size.

4. Bulk aqueous dispersion was further aseptically filtered, filled, and sealed in suitable container system such as glass vial stoppered by compatible rubber closure or Ampule or prefilled syringe.

5. The finished product as prepared in step 4 is to be stored/supplied in suitable secondary packaging material i.e., light protective tray/ mono carton.

### Stability Study

The batch filled in a prefilled glass syringe, stoppered with Flurotec laminated butyl rubber stopper was loaded in different stability conditions and the critical quality attributes such pH, assay, level of oxidative degradation products are assessed and summarized in Table 9.

TABLE 9

Stability study results of composition of formula 10

| Condition | Initial | 1 Month 25° C./ 60%RH | 1 Month 30° C. / 75%RH | 1 Month 40° C./ 75%RH |
|---|---|---|---|---|
| Description | Clear yellow solution | Clear yellow solution | | |
| pH | 5.43 | 5.34 | 5.35 | 5.32 |
| Assay (%) | 105.0 | 101.2 | 101.7 | 99.3 |
| Colour index (AU) | 0.031 | 0.044 | 0.048 | 0.077 |
| Degradation products (%) | | | | |
| Diol Impurity | ND | ND | <0.1 | <0.1 |
| Transepoxy Impurity | 0.201 | 0.135 | 0.135 | 0.146 |
| Hydroperoxide Impurity | <0.1 | <0.1 | <0.1 | <0.1 |
| Hydroxy Impurity | <0.1 | <0.1 | <0.1 | <0.1 |
| Total degradation products | 0.723 | 0.350 | 0.360 | 0.450 |
| Micellar Size (nm) | | | | |
| D10 | 8.56 | 8.74 | 8.76 | 8.72 |
| D50 | 11.4 | 11.6 | 11.7 | 11.7 |
| D90 | 15.8 | 16.3 | 16.5 | 17.0 |
| Span | 0.634 | 0.649 | 0.661 | 0.706 |
| Mean Droplet size (nm) | 11.82 | 12.07 | 12.14 | 12.30 |

### Observation:

It is observed that batches prepared with composition of present invention shows control on oxidative impurities and total impurities below the desired levels.

### Conclusion:

Based on the above result, it is clearly shown that the inclusion of preferred antioxidants improvised the stability of formulation in respect to degradation impurities.

The preparation of all the exemplified formulations were carried out under subdued light using sodium vapour lamp. The dissolved oxygen level in the formulation were controlled through nitrogen sparging of the solution during the compounding process (ex: to < 2 ppm) and the headspace of container was flushed with inert nitrogen gas prior to sealing.

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise. Thus, for example, reference to a “cosolvent” refers to a single cosolvent or to combinations of two or more cosolvents, and the like.

We claim:

1. A long-term storage stable pharmaceutical Phytonadione injectable formulation comprising:
a) Phytonadione or a pharmaceutically acceptable salt thereof;
b) an antioxidant;
c) a surfactant,
d) pharmaceutically acceptable excipients thereof.

2. The pharmaceutical formulation as claimed in claim 1, wherein the amount of phytonadione is from about 0.5 mg to about 20 mg per mL of total formulation.

3. The pharmaceutical formulation as claimed in claim 1, wherein the antioxidant comprises of of L-Methionine, L-Cysteine, thioglycerols, glutathione, ascorbic acid or salts thereof, ascorbyl palmitate, sodium metabisulfite, propyl gallate, butylated hydroxy anisole, butylated hydroxytoluene, tocopherol, histidine, amino acids or salts thereof, reducing sugars or any mixtures thereof, sodium citrate, trisodium citrate dihydrate, lactobionic acid, disodium ethylenediaminetetraacetic acid (EDTA), tetrasodium EDTA or combinations thereof.

4. The pharmaceutical formulation according to claim 1 wherein the antioxidant is L-Cysteine, Monothioglycerol or combination thereof.

5. The pharmaceutical formulation according to claim 1 wherein the antioxidant is present in an amount in the range from about 0.05 mg to about 10 mg per ml of the formulation.

6. The pharmaceutical formulation as claimed in claim 1, wherein the surfactant comprises of polysorbate 80, polyoxyl 35 castor oil mixed micelles comprising glycocholic acid and lecithin and combinations thereof.

7. The pharmaceutical formulation according to claim 1 wherein the surfactant is present in an amount from about 2 mg to about 100 mg per ml of the formulation.

8. The pharmaceutical formulation according to claim 1, wherein the excipients comprise of tonicity adjusting agents, buffering agent, pharmaceutically acceptable fluid and combination thereof.

9. The pharmaceutical formulation according to claim 1, wherein the buffering agent comprises of sodium acetate & glacial acetic acid and are present in an amount from about 0.05 mg to 1 mg per ml of formulation.

10. The pharmaceutical formulation according to claim 1, wherein the pharmaceutically acceptable fluid is selected from aqueous solution, water, saline, D5W, mixture of propylene glycol (PG) and water and combinations thereof.

11. The pharmaceutical formulation according to claim 1 wherein the pH of the formulation is from about 3.5 to 7.

12. The pharmaceutical formulation according to claim 1, wherein the stability of the formulation when stored for six months in a sealed and sterile vial at 25° C./ 60% RH and 40° C./ 75% RH contains no more than 1.0% of individual oxidative degradation products and no more than 3.0% of total degradation products as measured by HPLC.

13. A method of producing a pharmaceutical formulation comprising the steps of :
(a) dissolving phytonadione or pharmaceutically acceptable salt thereof in a pharmaceutically acceptable fluid to form a solution;
(b) adding an antioxidant, and a buffering agent to the solution; and
(c) adjusting the pH of the solution to from about 3.4 to about 7.0.

14. A method for treating malignant coagulation disorders comprising administering a pharmaceutical formulation according to claim 1 to a subject in need thereof.
